(19) United States
(12) Patent Application Publication
(10) Pub. No.: US 2025/0308627 A1
(43) Pub. Date: Oct. 2, 2025

(54) T-CELL RECEPTOR-PEPTIDE INTERACTION PREDICTION FOR MEDICAL DECISION MAKING

(71) Applicant: NEC Laboratories America, Inc., Princeton, NJ (US)

(72) Inventors: Renqiang Min, Princeton, NJ (US); Jonathan Warrell, Princeton, NJ (US); Tianxiao Li, Plainsboro, NJ (US)

(21) Appl. No.: 19/097,468

(22) Filed: Apr. 1, 2025

Related U.S. Application Data
(60) Provisional application No. 63/573,001, filed on Apr. 2, 2024.

(51) Int. Cl. G16B 15/30; G06Q 50/20; G16B 40/00

(52) U.S. Cl. CPC G16B 15/30; G06Q 50/20; G16B 40/00

(57) ABSTRACT

Methods and systems for tailored treatment include embedding a T-cell receptor (TCR) sequence and embedding an epitope sequence. The embedded TCR sequence and the embedded epitope sequence are processed with a discriminator to generate a multi-class label. The multi-class label is classified to generate a binary binding prediction. A treatment is generated based on the binary binding prediction.

### RELATED APPLICATION INFORMATION

This application claims priority to U.S. Patent Application No. 63/573,001, filed on Apr. 2, 2024, incorporated herein by reference in its entirety.

### BACKGROUND

### Technical Field

The present invention relates to T-cell receptor-peptide interaction prediction and, more particularly to the use of large language models for interaction prediction.

### Description of the Related Art

During the immune process, neoantigens and virus epitopes are presented by the major histocompatibility complex (MHC), which is then recognized by the T-cell receptor (TCR) on the surface of T CD8+ cells. Predicting this TCR-epitope binding event helps identify TCR molecules that can interact with specific target epitopes involved in disease processes. Predicting TCR-epitope binding events helps with target protein identification, drug discovery, the repurposing of existing drugs, and personalized medicine.

### SUMMARY

A method for tailored treatment includes embedding a T-cell receptor (TCR) sequence and embedding an epitope sequence. The embedded TCR sequence and the embedded epitope sequence are processed with a discriminator to generate a multi-class label. The multi-class label is classified to generate a binary binding prediction. A treatment is generated based on the binary binding prediction.

A system for tailored treatment includes a hardware processor and a memory that stores a computer program. When executed by the hardware processor, the computer program causes the hardware processor to embed a T-cell receptor (TCR) sequence, to embed an epitope sequence, to process the embedded TCR sequence and the embedded epitope sequence with a discriminator to generate a multi-class label, to classify the multi-class label to generate a binary binding prediction, and to generate a treatment based on the binary binding prediction.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram illustrating a framework for T-cell receptor (TCR)-epitope binding prediction, in accordance with an embodiment of the present invention;

FIG. 2 is a block diagram illustrating the operation of a discriminator in a TCR-epitope prediction model, in accordance with an embodiment of the present invention;

FIG. 3 is a block/flow diagram of a method for training and using a model for predicting binding between a TCR sequence and an epitope sequence, in accordance with an embodiment of the present invention;

FIG. 4 is a block diagram showing a healthcare facility where TCR-epitope binding prediction can be used to tailor treatments, in accordance with an embodiment of the present invention;

FIG. 5 is a block diagram of a computing device that can train and use a model for predicting binding between a TCR sequence and an epitope sequence, in accordance with an embodiment of the present invention;

FIG. 6 is a diagram of an exemplary neural network architecture that can be used to implement part of a binding prediction classifier, in accordance with an embodiment of the present invention; and

FIG. 7 is a diagram of an exemplary deep neural network architecture that can be used to implement part of a binding prediction classifier, in accordance with an embodiment of the present invention.

### DETAILED DESCRIPTION OF PREFERRED EMBODIMENTS

A large language model (LLM) can be used to predict T-cell receptor (TCR)-epitope binding events. A pretrained LLM may be used as a backbone model, and TCR sequences can then be used to tune the backbone model via masked language modeling. After the tuning, the LLM may be used to extract features of the TCR sequences.

Embeddings of epitope sequences may be obtained from the pre-trained LLM. Each data entry may include embeddings of a CDR3A, a CDR3B, an epitope, and a label for whether the set is binding or non-binding. The data may then be input to a discriminator to refine the embeddings. The discriminator may have three transformer-based first-level encoders for the CDR3A, CDR3B, and epitope inputs, and may further have a multilayer perceptron (MLP)-based classifier which accepts the output of the second-level encoder to predict the epitope label. The discriminator engages in a multi-class classification task to enhance the distinguishability of TCR sequences for each epitope and then to facilitate the classification. Alternatively, a discriminator may include encoders for only the CDR3BN and epitope. The output of the discriminator may be concatenated embeddings of the pairs. These embeddings may be input to the MLP-based classifier for binary classification, indicating whether the pair is binding or non-binding.

This LLM-based approach takes full advantage of the available data and labels, and uses transfer learning on the pretrained LLM for TCR-epitope binding prediction. The result is a model that provides strong predictive accuracy.

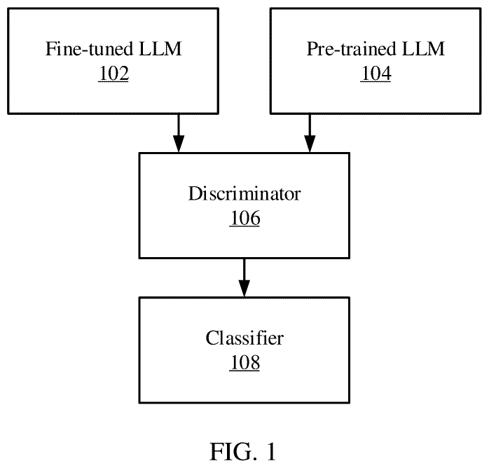

Referring now to FIG. 1, a block diagram of a binding prediction framework is shown. A fine-tuned LLM 102 is used to extract features of TCR sequences and a pre-trained LLM 104 is used to extract features of epitope sequences. As will be described in greater detail below, in some cases the pre-trained LLM 104 may be the same base LLM as the fine-tuned LLM 102, before fine-tuning, but the pre-trained LLM 104 may alternatively be a different model.

A discriminator 106 is used to refine the input embeddings using transformer-based first-level encoders, a second-level encoder, and one or more MLPs. The output of the discriminator 106 may be a concatenated embedding of, e.g., a (CDR3B, epitope) tuple or a (CDR3A, CDR3B, epitope) tuple. A classifier 108, for example based on MLPs, may be used to generate a binary classifier to determine whether the tuple is binding or non-binding.

With the extraordinary performance of LLMs in understanding and generating language with near-human capabilities, LLMs open a new door to address TCR-epitope binding problems from sequence data. The TCR sequence patterns are integrated in a hierarchical manner highly analogous to human languages: the letters (e.g., amino acids) are arranged to form secondary structural elements (“words”), which assemble to form domains (“sentences”) that undertake a function (“meaning”). TCR sequences are information-complete: they store structure and function entirely in their amino acid bases and order with extreme efficiency. Given the abundance of TCR sequences available from public databases, but with limited availability of epitope-TCR binding data, a viable approach is to train the model in a transfer learning manner. Specifically, an LLM may be trained by masked language modeling using unlabeled TCR data. This enables the model to grasp the inherent and general patterns within the sequences. Subsequently, the pretrained model can be fine-tuned on the TCR-epitope binding data for the classification task (binding or non-binding) or other tasks.

The present framework uses the pretrained LLM 104 as the backbone model. An exemplary pretrained LLM may be protBERT. Other exemplary backbones include TCR-BERT and ESM. The protBERT and ESM models are pretrained by protein sequences, while TCR-BERT is pretrained specifically with TCR sequences.

TCR sequences are used to tune the backbone model via masked language modeling. In one specific example, the pretrained model 104 may be tuned using a set of TCR sequences, including some TCR3B sequences and some TCR3A sequences. After masked language modeling tuning, the fine-tuned LLM 102 extracts features of the TCR sequences. For the epitope sequences, the pre-trained LLM model 104 is used to extract embeddings.

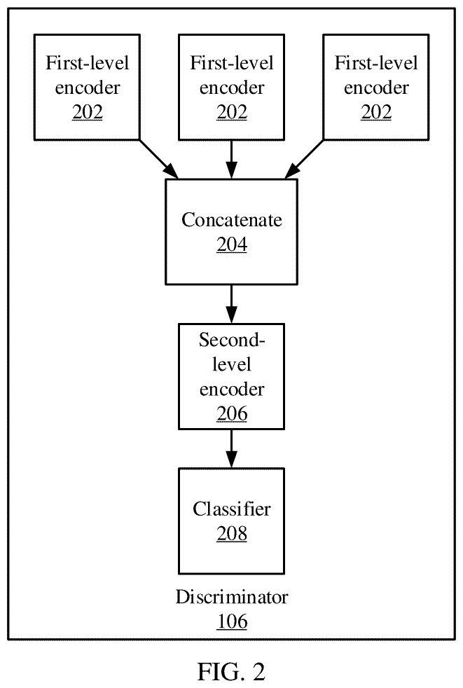

Referring now to FIG. 2, additional detail on the discriminator 106 is shown. The discriminator 106 has three transformer-based first-level encoders 2022 for three inputs (CDR3A, CDR3B, and epitope), a transformer-based second-level encoder 206 for the concatenated outputs of the first-level encoders. An MLP-based classifier 208 accepts the output of the second-level encoder 206.

The aim of this discriminator 106 is to engage in a multi-class classification task, intending to enhance the distinguishability of TCR sequences for each epitope and then facilitate the classification. The embeddings from the discriminator 106 are input to an MLP-based prediction classifier 108.

The discriminator 106 makes TCR sequences of different epitopes distinguishable for the classifier 108. During training, discriminator 106 can see the paired epitopes of the TCRs (CDR3A and CDR3B) but cannot see the binding information between them (0 or 1). The embeddings of the three sequences (CDR3A, CDR3B, and epitope) are used as the inputs for discriminator 106. Stacked transformer layers as the first-level encoders 202 for each input sequence. Formally, the outputs of the first-level encoders 202 can be shown as:

$${E}_{a}^{′} = {TF}_{a}^{1} ( {E}_{a} )$$

$${E}_{b}^{′} = {TF}_{b}^{1} ( {E}_{b} )$$

$${E}_{e}^{′} = {TF}_{e}^{1} ( {E}_{e} )$$

where Eₐ, E_{b}, and Eₑ respectively are embeddings for the CDR3A sequence, the CDR3B sequence, and the epitope sequence, and where

$${TF}_{a}^{1} , {TF}_{b}^{1} , and {TF}_{e}^{1}$$

respectively are first-level transformer-based encoders 202 for the CDR3A sequence, the CDR3B sequence, and the epitope sequence.

The outputs of the first-level encoders 202 may be concatenated 204 as the input for a second-level encoder 206, which may also be implemented with stacked transformer layers TF²:

$$E = {E}_{a}^{′} ⊕ {E}_{b}^{′} ⊕ {E}_{e}^{′}$$

$${E}^{′} = {TF}^{2} ( E )$$

The dimension of E and E′ may thus be three times the dimension of the output of the first-level encoders 202, with no reduction in dimension by the second-level encoder. In embodiments that instead (CDR3B, epitope) tuples, the output dimension of the second-level encoder 206 may be twice that of the first-level encoders 202.

The discriminator 106 then uses an MLP-based classifier 208 for the output of the second-level encoder 206 to recover the dimension of the inputs and to build a reconstruction loss between each input and the corresponding reconstructed output:

$${E}_{a}^{r e} = {D}_{a} ( {E}^{′} )$$

$${E}_{b}^{r e} = {D}_{b} ( {E}^{′} )$$

$${E}_{e}^{r e} = {D}_{e} ( {E}^{′} )$$

$${L}_{a}^{r e} = m s e ( {E}_{a}^{r e} , {E}_{a} )$$

$${L}_{b}^{r e} = m s e ( {E}_{b}^{r e} , {E}_{b} )$$

$${L}_{e}^{r e} = m s e ( {E}_{e}^{r e} , {E}_{e} )$$

where

$${E}_{a}^{r e} , {E}_{b}^{r e} , and {E}_{e}^{r e} ∈ {N \times D}^{}$$

stand for the reconstructed embedding of CDR3A, CDR3B and epitope, and Dₐ, D_{b}, and Dₑ are the decoders for them, where N is the number of input samples and D is the dimension of the embedding. The mse(.) function indicates the mean square error and

$${L}_{a}^{r e} , {L}_{b}^{r e} , and {L}_{e}^{r e}$$

stand for the reconstruction loss for CDR3A, CDR3B and epitope, respectively. These reconstruction losses are used to prevent the information learned from LLM from being impaired during the training of discriminator 106. Then, in the latent space, the MLP classifier 208 is used to reduce the dimension of the embeddings into the number of unique epitopes in the data and do a multi-class classification using the epitopes as the labels.

$${E}^{″} = MLP ( {E}^{′} )$$

$${L}^{c l s} = CE ( {E}^{″} , {S}_{e} )$$

where CE(⋅) indicates the cross-entropy loss function for multi-class classification and MLP is one fully connected layer to reduce the dimensions of the concatenated embeddings into the number of unique epitopes (e). E″∈ [symbol] ^{N×e} is the output of the MLP classifier 208, where e indicates the number of unique epitopes in the data.

In an exemplary embodiment, the first-level encoders 202 may use four heads and four layers in their transformer blocks. In such an exemplary embodiment, the second-level encoder 206 may have eight heads and four layers. The MLP classifier 108 may have three layers with dimensions [256, 64, 1]. The dropout rates may be set to 0.1 in the transformer layers and to 0.3 in the MLP layers. Rectified linear units (ReLU) may be used for the activation function of the transformers and the MLP layers. The output dimension of the first-level encoders 202 may be set to 256. The dimension of the concatenated latent space may therefore be 768. These dropout rates and activations may be applied to the MLPs in both the binary classifier and in the discriminator.

The dimension of the input sequences is reduced in the feed-forward layer of the last stacked layer in the first-level encoders 202, preventing the dimension of the concatenated output of the first level encoders 202 to be too high. A ResNet therefore cannot be used on the last layer of the first-level encoders 202. In the case where the dimension of x equals the dimension of ff(x):

$$x = norm ( norm ( x + att ( x , mask ) ) + ff ( norm ( x + att ( x , mask ) ) ) )$$

$$Otherwise :$$

$$x = norm ( ff ( norm ( x + att ( x , mask ) ) ) )$$

where dim(⋅) outputs the dimension of the input or output vectors, ff(⋅) is the feed-forward block, and norm(⋅) is a layer normalization function. The value mask stands for the mask function for masked language modeling. The adjusted transformer layer has a similar performance to the original one but has advantages on scalability especially when the embeddings from the LLM has a high dimension. The total loss of the discriminator 106 is:

$$L = {w}_{cls} \cdot {L}^{cls} + {L}_{a}^{r e} + {L}_{b}^{r e} + {L}_{e}^{r e}$$

where w_{cls} is the weight for the multi-class classification loss L^{cls}, which may be set to 0.1 by default.

The output of the discriminator 106 may then be classified by another MLP-based classifier 108 to produce a binary classification for binding prediction:

$${E}^{′′′} = {MLP}^{binary} ( {E}^{″} )$$

$${L}^{binary} = BCE ( sigmoid ( {E}^{′′′} ) , y )$$

where E″′∈ [symbol] ^{N×1} stands for the output of the MLP layers of classifier 108, BCE(⋅) is the binary cross-entropy loss, y is the binary (0 and 1) labels indicating if the pairs can bind or not, and sigmoid(⋅) is the activation function to non-linearly transform the outputs from MLP layers. All the pairs of data are used as inputs into the discriminator to update their embeddings. However, the classifier model is trained by a training subset, with the training process being monitored using a validation subset, and with model performance being monitored using a testing data subset. The discriminator 106 DC and classifier 108 are not trained simultaneously, so the parameters of the discriminator 106 may be fixed when classifier 108 is being trained.

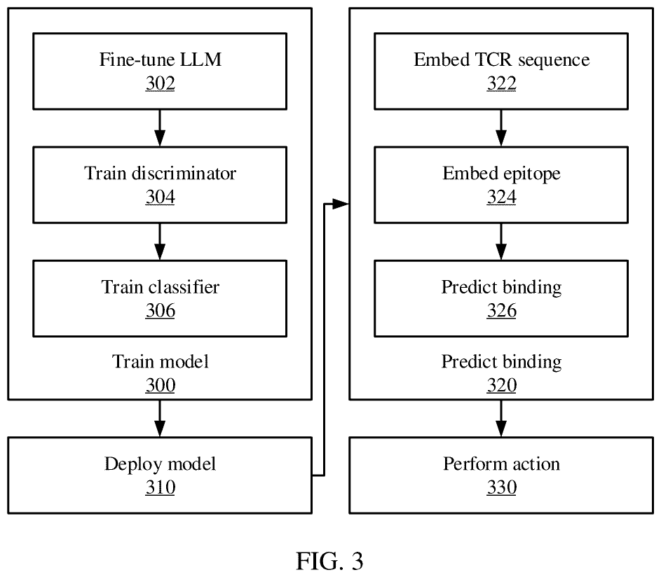

Referring now to FIG. 3, a method for training and using a binding prediction model is shown. The model is trained by block 300, which includes fine-tuning 302 the pretrained LLM 104 using masked language modeling with the TCR sequences. The fine-tuning 302 may use an exemplary batch size of 8 and an exemplary learning rate of 1·10⁻⁶. Block 304 trains the discriminator 106 and block 306 trains the classifier 108, with exemplary batch sizes of 64 and exemplary learning rates of 1·10⁻⁶.

Training 304 of the discriminator 106 may stop when a training loss has converged or when a maximum number of training epochs (e.g., 400) has been reached. An early-stop function may be used with delta=0 and patience=5. For the training 306 of the classifier 108, an early stop function may monitor a change of the validation loss. The patience in that case may be set to 5 and the delta may be set to 0.005.

Once trained, the model may be deployed in block 310. This deployment may include copying the parameters of the trained models to a target system, where new TCR information may be available for binding prediction. In cases where the model is going to be used by the same system that trains it, the deployment 310 may be omitted.

Block 320 predicts binding between a TCR sequence and an epitope. Block 322 embeds a TCR sequence, such as a CDR3A and/or CDR3B sequence, for example from a database of such sequences, using the fine-tuned LLM 102. Block 324 embeds a new epitope sequence, for example using the pre-trained LLM 104. These embeddings are combined in the discriminator 106 as described above in block 326, which then uses the classifier 108 to predict whether the epitope binds to the TCR sequence(s).

Based on the binding prediction, block 330 performs an action. In some embodiments, this action may include the production of a tailored therapy for a patient. Adoptive T-cell immunotherapy is an example of such an action, where autologous T-cells are taken from a patient and are genetically modified to bind to cancer cells in the patient's body. The modified T-cells are infused back into the patient. TCR T-cell therapy directly modifies the TCRs of T-cells to increase their binding affinities, which makes it possible to recognize and kill tumor cells.

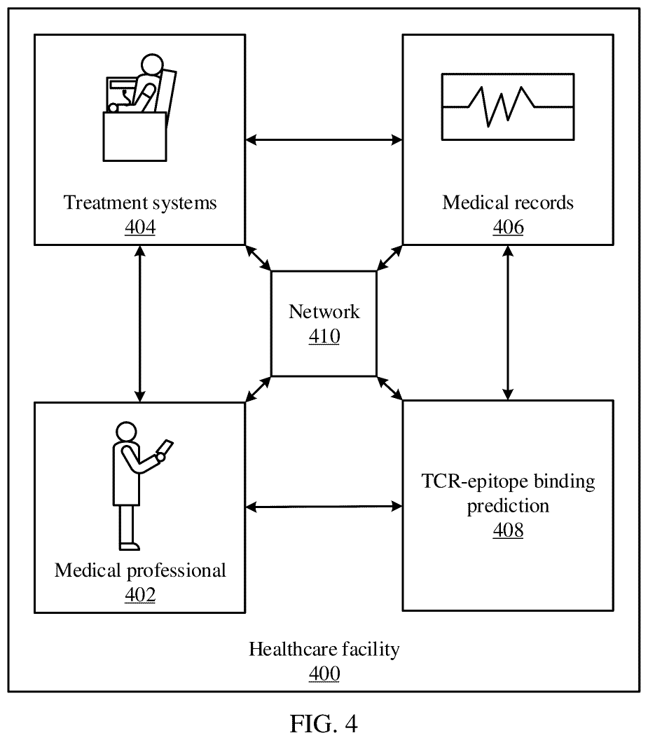

Referring now to FIG. 4, a diagram of therapy generation is shown in the context of a healthcare facility 400. TCR-epitope binding prediction 408 may be used to generate a custom treatment for a patient, based on the determination that the immune system will respond to a given sequence. TCR-epitope binding prediction 408 may be used to generate a treatment responsive to a patient's medical condition based on up-to-date medical records 406.

The healthcare facility may include one or more medical professionals 402 who review information extracted from a patient's medical records 406 to determine their healthcare and treatment needs. These medical records 406 may include self-reported information from the patient, test results, and notes by healthcare personnel made to the patient's file. Treatment systems 404 may furthermore monitor patient status to generate medical records 406 and may be designed to automatically administer and adjust treatments as needed.

Medical professionals 402 may use TCR-epitope binding prediction 408 to provide customized healthcare that is tailored to the patient's needs. For example, the medical professionals 402 may use TCR-epitope binding prediction 408 to generate a new drug that will cause an immune system response, for example to build immune system defenses against a particular disease.

The different elements of the healthcare facility 400 may communicate with one another via a network 410, for example using any appropriate wired or wireless communications protocol and medium. Thus the TCR-epitope binding prediction 408 can be used to design a treatment that targets a patient's specific condition, for example using test results and medical records 406. The treatment systems 404 may be used to generate and administer a therapy based on TCR-epitope binding prediction 408.

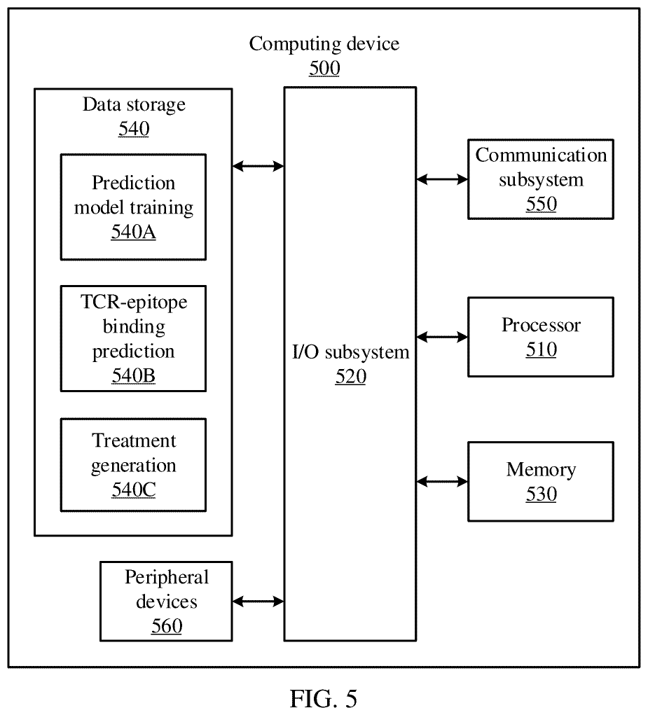

As shown in FIG. 5, the computing device 500 illustratively includes the processor 510, an input/output subsystem 520, a memory 530, a data storage device 540, and a communication subsystem 550, and/or other components and devices commonly found in a server or similar computing device. The computing device 500 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 530, or portions thereof, may be incorporated in the processor 510 in some embodiments.

The processor 510 may be embodied as any type of processor capable of performing the functions described herein. The processor 510 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 530 may be embodied as any type of volatile or non-volatile memory or data storage capable of performing the functions described herein. In operation, the memory 530 may store various data and software used during operation of the computing device 500, such as operating systems, applications, programs, libraries, and drivers. The memory 530 is communicatively coupled to the processor 510 via the I/O subsystem 520, which may be embodied as circuitry and/or components to facilitate input/output operations with the processor 510, the memory 530, and other components of the computing device 500. For example, the I/O subsystem 520 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 520 may form a portion of a system-on-a-chip (SOC) and be incorporated, along with the processor 510, the memory 530, and other components of the computing device 500, on a single integrated circuit chip.

The data storage device 540 may be embodied as any type of device or devices configured for short-term or long-term storage of data such as, for example, memory devices and circuits, memory cards, hard disk drives, solid state drives, or other data storage devices. The data storage device 540 can store program code 540A for training a prediction model, 540B for performing TCR-epitope binding prediction, and/or 540C for generating a treatment. Any or all of these program code blocks may be included in a given computing system. The communication subsystem 550 of the computing device 500 may be embodied as any network interface controller or other communication circuit, device, or collection thereof, capable of enabling communications between the computing device 500 and other remote devices over a network. The communication subsystem 550 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 500 may also include one or more peripheral devices 560. The peripheral devices 560 may include any number of additional input/output devices, interface devices, and/or other peripheral devices. For example, in some embodiments, the peripheral devices 560 may include a display, touch screen, graphics circuitry, keyboard, mouse, speaker system, microphone, network interface, and/or other input/output devices, interface devices, and/or peripheral devices.

Of course, the computing device 500 may also include other elements (not shown), as readily contemplated by one of skill in the art, as well as omit certain elements. For example, various other sensors, input devices, and/or output devices can be included in computing device 500, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 500 are readily contemplated by one of ordinary skill in the art given the teachings of the present invention provided herein.

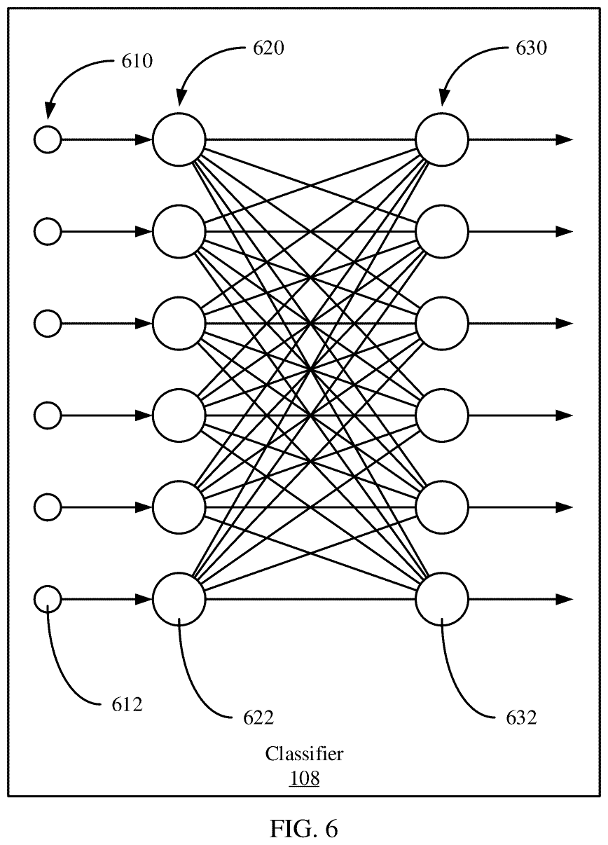

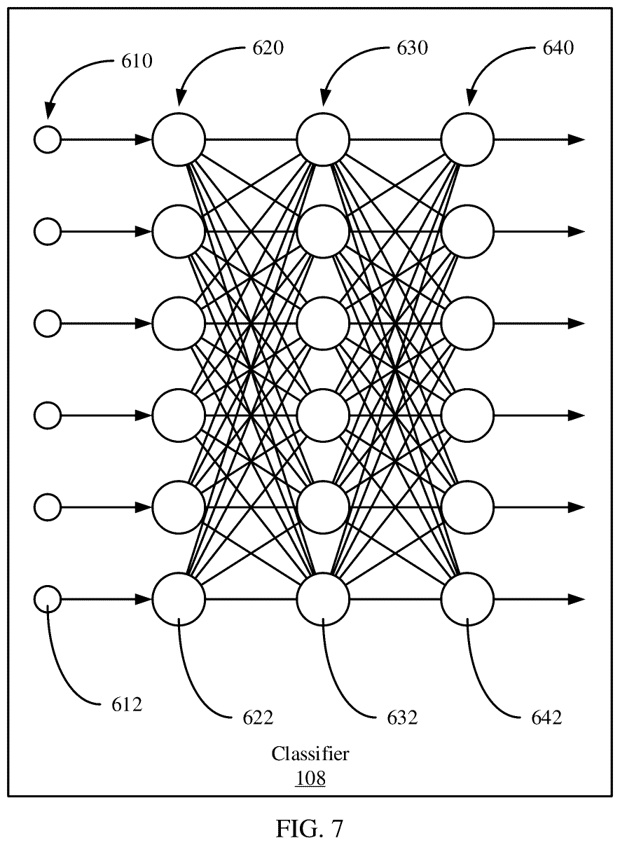

Referring now to FIGS. 6 and 7, exemplary neural network architectures are shown, which may be used to implement parts of the present models, such as the classifier 108. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 620 of source nodes 622, and a single computation layer 630 having one or more computation nodes 632 that also act as output nodes, where there is a single computation node 632 for each possible category into which the input example could be classified. An input layer 620 can have a number of source nodes 622 equal to the number of data values 612 in the input data 610. The data values 612 in the input data 610 can be represented as a column vector. Each computation node 632 in the computation layer 630 generates a linear combination of weighted values from the input data 610 fed into input nodes 620, and applies a non-linear activation function that is differentiable to the sum. The exemplary simple neural network can perform classification on linearly separable examples (e.g., patterns).

A deep neural network, such as a multilayer perceptron, can have an input layer 620 of source nodes 622, one or more computation layer(s) 630 having one or more computation nodes 632, and an output layer 640, where there is a single output node 642 for each possible category into which the input example could be classified. An input layer 620 can have a number of source nodes 622 equal to the number of data values 612 in the input data 610. The computation nodes 632 in the computation layer(s) 630 can also be referred to as hidden layers, because they are between the source nodes 622 and output node(s) 642 and are not directly observed. Each node 632, 642 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 632 in the one or more computation (hidden) layer(s) 630 perform a nonlinear transformation on the input data 612 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor-or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for tailored treatment, comprising:
embedding a T-cell receptor (TCR) sequence;
embedding an epitope sequence;
processing the embedded TCR sequence and the embedded epitope sequence with a discriminator to generate a multi-class label;
classifying the multi-class label to generate a binary binding prediction; and
generating a treatment based on the binary binding prediction.

2. The method of claim 1, wherein the epitope sequence is embedded using a pre-trained large language model (LLM).

3. The method of claim 2, wherein the TCR sequence is embedded using a separate version of the pre-trained LLM that has been fine-tuned on TCR sequences.

4. The method of claim 1, wherein the discriminator includes a plurality of first-level transformer-based encoders.

5. The method of claim 4, wherein the TCR sequence includes a CDR3A sequence and a CDR3B sequence that are processed by different respective first-level transformer-based encoders.

6. The method of claim 4, wherein the discriminator further includes a second-level transformer-based encoder that accepts as input a combination of the outputs of the first-level transformer-based encoders.

7. The method of claim 6, wherein the discriminator further includes a multilayer perceptron (MLP)-based classifier that accepts as input the output of the second-level transformer-based encoder and that outputs the multi-class label.

8. The method of claim 4, wherein the outputs of the first-level transformer-based encoders are concatenated to generate the input of the second-level transformer-based encoder.

9. The method of claim 1, wherein the embedding, processing, and classifying are performed using a machine learning model.

10. The method of claim 1, wherein the binding prediction is used by medical professionals to aid in medical decision-making regarding use of the treatment to treat a patient.

11. A system for tailored treatment, comprising:
a hardware processor; and
a memory that stores a computer program which, when executed by the hardware processor, causes the hardware processor to:
embed a T-cell receptor (TCR) sequence;
embed an epitope sequence;
process the embedded TCR sequence and the embedded epitope sequence with a discriminator to generate a multi-class label;
classify the multi-class label to generate a binary binding prediction; and
generate a treatment based on the binary binding prediction.

12. The system of claim 11, wherein the epitope sequence is embedded using a pre-trained large language model (LLM).

13. The system of claim 12, wherein the TCR sequence is embedded using a separate version of the pre-trained LLM that has been fine-tuned on TCR sequences.

14. The system of claim 11, wherein the discriminator includes a plurality of first-level transformer-based encoders.

15. The system of claim 14, wherein the TCR sequence includes a CDR3A sequence and a CDR3B sequence that are processed by different respective first-level transformer-based encoders.

16. The system of claim 14, wherein the discriminator further includes a second-level transformer-based encoder that accepts as input a combination of the outputs of the first-level transformer-based encoders.

17. The system of claim 16, wherein the discriminator further includes a multilayer perceptron (MLP)-based classifier that accepts as input the output of the second-level transformer-based encoder and that outputs the multi-class label.

18. The system of claim 14, wherein the outputs of the first-level transformer-based encoders are concatenated to generate the input of the second-level transformer-based encoder.

19. The system of claim 11, wherein the embedding, processing, and classifying are performed using a machine learning model.

20. The system of claim 11, wherein the binding prediction is used by medical professionals to aid in medical decision-making regarding use of the treatment to treat a patient.
